SEQUENCE LISTING (8 sequences)

SEQ ID NO: 1 - protein, 9 aa, Homo sapiens
KKLQDVHNF

SEQ ID NO: 2 - protein, 5 aa, Homo sapiens
LQDVH

SEQ ID NO: 3 - protein, 84 aa, Homo sapiens
SVSEIQLMHNLGKHLNSMERVEWLRKKLQDVHNFVALGAPLAPRDAGSQRPRKKEDNVLVESHEKSLGEANKADVNVLTKAKSQ

SEQ ID NO: 4 - protein, 83 aa, Homo sapiens
VSEIQLMHNLGKHLNSMERVEWLRKKLQDVHNFVALGAPLAPRDAGSQRPRKKEDNVLVESHEKSLGEANKADVNVLTKAKSQ

SEQ ID NO: 5 - protein, 51 aa, Homo sapiens
FVALGAPLAPRDAGSQRPRKKEDNVLVESHEKSLGEANKADVNVLTKAKSQ

SEQ ID NO: 6 - protein, 34 aa, Homo sapiens
SVSEIQLMHNLGKHLNSMERVEWLRKKLQDVHNF

SEQ ID NO: 7 - protein, 50 aa, Homo sapiens
VALGAPLAPRDAGSQRPRKKEDNVLVESHEKSLGEANKADVNVLTKAKSQ

SEQ ID NO: 8 - protein, 20 aa, Homo sapiens
KSLGEANKADVNVLTKAKSQ

(19) United States
(12) Patent Application Publication
(10) Pub. No.: US 2003/0138858 A1
(43) Pub. Date: Jul. 24, 2003

(54) Methods and devices for direct determination of cyclase inhibiting parathyroid hormone

(76) Inventor: Thomas L. Cantor, El Cajon, CA (US)

Correspondence Address: Peng Chen, Morrison & Foerster LLP, 3811 Valley Centre Drive, Suite 500, San Diego, CA, 92130-2332, US

(21) Appl. No.: 09/928,048

(22) Filed: Aug. 10, 2001

Related U.S. Application Data
(60) Provisional application No. 60/224,447, filed on Aug. 10, 2000.

(51) Int. Cl. G01N 33/53; G01N 33/542; C07K 16/26

(52) U.S. Cl. 435/7.9; 530/388.24

(57) ABSTRACT

The present invention relates to novel methods and devices for directly determining the presence or amount of cyclase inhibiting parathyroid hormone present in a clinical sample. Such determinations are useful in differentiating parathyroid diseases, such as hyperparathyroidism, from normal or non-disease states. The target analyte is a large non-whole parathyroid hormone peptide fragment that can function as a cyclase activating parathyroid hormone antagonist.

### TECHNICAL FIELD

The present invention relates to novel methods and devices for directly determining the presence or amount of cyclase inhibiting parathyroid hormone present in a clinical sample. Such determinations are useful in differentiating parathyroid diseases, such as hyperparathyroidism, from normal or non-disease states. The target analyte is a large non-whole parathyroid hormone peptide fragment that can function as a cyclase activating parathyroid hormone antagonist.

### BACKGROUND ART

Calcium plays an indispensable role in cell permeability, the formation of bones and teeth, blood coagulation, transmission of nerve impulse, and normal muscle contraction. The concentration of calcium ions in the blood is, along with calcitrol and calcitonin, regulated mainly by parathyroid hormone (PTH). Although calcium intake and excretion may vary, PTH serves through a feedback mechanism to maintain a steady concentration of calcium in cells and surrounding fluids. When serum calcium lowers, the parathyroid glands secrete PTH, affecting the release of stored calcium. When serum calcium increases, stored calcium release is retarded through lowered secretions of PTH.

The complete form of human PTH, sometimes referred to in the art as hPTH but referred to in the present invention either as whole PTH or CAP, is a unique 84 amino acid peptide (SEQ ID NO.3), as is shown in FIGURE 1. Researchers have found that this peptide has an anabolic effect on bone that involves a domain for protein kinase C activation (amino acid residues 28 to 34) as well as a domain for adenylate cyclase activation (amino acid residues 1 to 7). However, various catabolic forms of clipped or fragmented PTH peptides also are found in circulation, most likely formed by intraglandular or peripheral metabolism. For example, whole PTH can be cleaved between amino acids 34 and 35 to produce a (1-34) PTH N-terminal fragment and a (35-84) PTH C-terminal fragment. Likewise, clipping can occur between either amino acids 36 and 37 or 37 and 38. Recently, a large PTH fragment referred to as “non-(1-84) PTH” has been disclosed which is clipped closer to the N-terminal end of PTH. (See R. LePage et alia, “A non-(1-84) circulating parathyroid hormone (PTH) fragment interferes significantly with intact PTH commercial assay measurements in uremic samples” Clin Chem (1998); 44: 805-810.)

The clinical need for accurate measurement of PTH is well demonstrated. Serum PTH level is one of the most important indices for patients with the following diseases: familial hypocalciuria; hypercalcemia; multiple endocrine neoplasia types I and II; osteoporosis; Paget's bone disease; primary hyperparathyroidism caused by primary hyperplasia or adenoma of the parathyroid glands; pseudohypoparathyroidism; and renal failure, which can cause secondary hyperparathyroidism.

PTH plays a role in the course of disease in a patient with chronic renal failure. Renal osteodystrophy (RO) is a complex skeletal disease comprising osteitis fibrosa cystica (caused by PTH excess), osteomalacia resulting in unmineralized bone matrix (caused by vitamin D deficiency), extraskeletal calcification/ossification (caused by abnormal calcium and phosphorus metabolism), and adynamic bone disease (contributed to by PTH suppression). Chronic renal failure patients can develop RO. Failing kidneys increase serum phosphorus (hyperphosphoremia) and decrease 1,25-dihydroxyvitamin D (1,25-D) production by the kidney. The former results in secondary hyperparathyroidism from decreased gastrointestinal calcium absorption and osteitis fibrosa cystica from increased PTH in response to an increase in serum phosphorus. The later causes hypocalcemia and osteomalacia. With the onset of secondary hyperparathyroidism, the parathyroid gland becomes less responsive to its hormonal regulators because of decreased expression of its calcium and vitamin D receptors. Serum calcium drops. RO can lead to digital gangrene, bone pain, bone fractures, and muscle weakness.

Determining circulating biologically active PTH levels in humans has been challenging. One major problem is that PTH is found at low levels, normally 10 pg/mL to 65 pg/mL. Coupled with extremely low circulating levels is the problem of the heterogeneity of PTH and its many circulating fragments. In many cases, immunoassays have faced substantial and significant interference from circulating PTH fragments. For example, some commercially available PTH kits have almost 100% cross-reactivity with the non-(1-84) PTH fragment, (see the LePage article).

PTH immunoassays have varied over the years. One early approach is a double antibody precipitation immunoassay found in U.S. Pat. No. 4,369,138 to Arnold W. Lindall et alia A first antibody has a high affinity for a (65-84) PTH fragment. A radioactive labeled (65-84) PTH peptide is added to the sample with the first antibody to compete for the endogenous unlabeled peptide. A second antibody is added which binds to any first antibody and radioactive labeled PTH fragment complex, thereby forming a precipitate. Both precipitate and supernatant can be measured for radioactive activity, and endogenous PTH levels can be calculated there from.

In an effort to overcome PTH fragment interference, immunoradiometric two-site assays for intact PTH (I-PTH) have been introduced, such as the Allegro® Intact PTH assay by the Nichol's Institute of San Juan Capistrano, Calif. In one version, a capture antibody specifically binds to the C-terminal portion of hPTH while another labeled antibody specifically binds to the N-terminal portion of the captured hPTH. In another assay, two monoclonal antibodies were used, both of which attached to the N-terminal portion of hPTH. Unfortunately, these assays have problems in that they measure but do not discriminate between whole PTH and non-whole PTH peptide fragments. This inability comes to the fore in hyperparathyroid patients and renal failure patients who have significant endogenous concentrations of large, non-whole PTH fragments.

Recently, researchers have made a specific binding assay directed to the large N-terminal PTH fragments. (See. Gao, Ping et alia “Immunochemicalluminometric assay with two monoclonal antibodies against the N-terminal sequence of human parathyroid hormone”, Clinica Chimica Acta 245 (1996) 39-59.) This immunochemiluminometric assay uses two monoclonal antibodies to detect N-terminal (1-34) PTH fragments but not mid-portion PTH fragments or C-terminal PTH fragments. A key factor in the design of these assays is to eliminate any reaction with C-terminal PTH fragments.

### DISCLOSURE OF THE INVENTION

The present invention relates to novel methods and devices for directly determining the presence or amount of cyclase inhibiting parathyroid hormone present in a clinical sample. Such determinations are useful in differentiating parathyroid diseases, such as hyperparathyroidism, from normal or non-disease states. The target analyte is a large non-whole parathyroid hormone peptide fragment that can function as a cyclase activating parathyroid hormone antagonist. For the purposes of the present invention, what is commonly referred to as PTH (the peptide having 84 amino acids) is referred to as cyclase activating PTH (CAP).

The present invention incorporates a discovery that a large, non-whole PTH peptide fragment, a peptide having an amino acid sequence from between (SEQ ID No.4 [PTH₂₋₈₄]) and (SEQ ID No.5 [PTH₃₄₋₈₄]), functions in vivo as an antagonist of CAP. In other words, the binding of CAP to PTH receptors and the subsequent biological activity are affected by the presence of this cyclase inhibiting PTH peptide fragment, referred to herein as CIP. The PTH receptors can be tied up with respect to CAP or CAP analogs in that the PTH binding site is blocked by CIP. The relationship between the concentrations of CAP and CIP vary with PTH related disease states, and thus, are indicative of such states. Equally useful in view of the discovery of the antagonistic nature of CIP, the present invention relates to novel methods and devices for monitoring parathyroid related bone diseases, and resultant bone loss or build-up. Increased amounts of CIP can inhibit the calcium releasing activity of CAP or the net biological activity that a given amount of CAP may have.

In making a direct measurement of CIP, one can use an antibody or antibody fragment specific for a peptide sequence for CIP which by virtue of the unique CIP protein conformation is available for antibody binding but this same epitope is not available for antibody binding in CAP by virtue of the unique CAP protein conformation of CAP, in an amount sufficient to bind the CIP present, and thus, enable immunoassay measurement. In other words, conformational changes between CAP and CIP do not make the CIP binding site available on CAP. Such a domain has been identified that functions in the opposite manner. The domain of PTH 28-32 is an epitope that is available for antibody binding on CAP, but not on CIP due to the unique protein conformational differences between CAP and CIP. Such an antibody or antibody fragment can be used in conventional immunoassay formats either as a signal antibody or a capture antibody. Such antibodies can be either monoclonal or polyclonal in nature.

To differentiate between parathyroid disease states and the normal state or to monitor the effects of therapeutic treatment for parathyroid disease states, one can compare the relationship between the values of CAP and CIP. For patients with a traditional “intact” PTH value over about 100 pg/ml, one should determine the CIP value, as the CAP to CIP ratio value changes significantly between a normal person and a patient with a parathyroid disease and between various stages of parathyroid diseases.

### BRIEF DESCRIPTION OF THE DRAWINGS

FIGURE 1 is a diagrammatic view of human CAP.

### BEST MODES FOR CARRYING OUT THE INVENTION

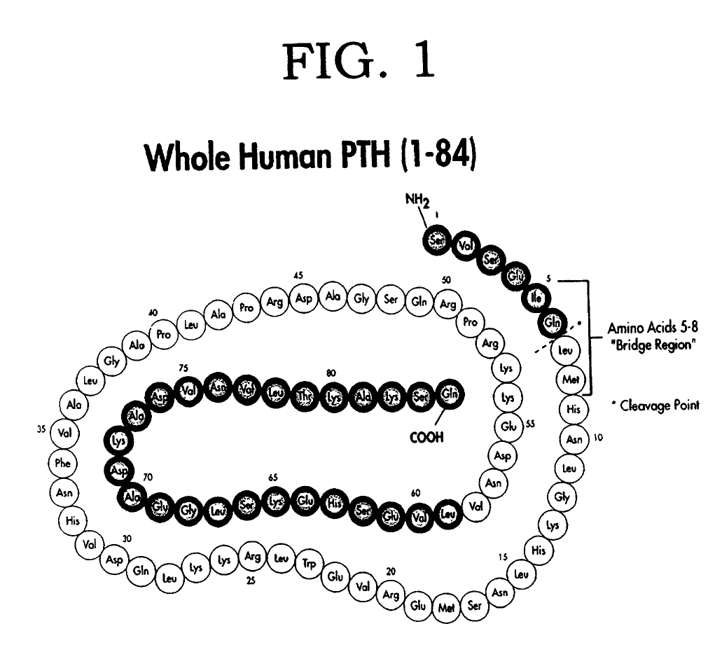

In disclosing the present invention, one should remember that there are a number of closely analogous, species dependent forms of PTH. The amino acid sequence of hPTH is shown in FIGURE 1. However, for rat PTH, bovine PTH, or porcine PTH, for example, one finds the substitutions at some of the amino acids in the hPTH sequence. For the purposes of the present invention, one can use interchangeably antibodies or antibody fragments to forms of these PTH's, although it is preferred to use an antibody with specificity for PTH having a sequence matching the species in which the PTH measurements are made.

Cyclase Inhibiting PTH Immunoassays

A first method for determining CIP in a sample comprises three basic steps. First one adds to the sample a labeled monoclonal or polyclonal antibody or antibody fragment specific for a peptide sequence for CIP that by virtue of the unique CIP protein conformation is available for antibody binding but this same epitope is not available for antibody binding in CAP by virtue of the unique protein conformational differences between CAP and CIP, in an amount sufficient to bind the CIP present, and thus, enable immunoassay measurement. Preferably, this domain does not comprises an amino acid sequence PTH₂₆₋₃₄ (SEQ ID No. 1), preferably PTH₂₈₋₃₂ (SEQ ID No. 2). Conditions for such reactions are well know to those of ordinary skill in the art. Second, one allows the labeled antibody to bind to any CIP present, thereby forming a complex. Third, one measures the amount of labeled complex.

One can modify this first method by adding a second antibody bound to a solid support and that specifically binds to a portion of CIP other than that of the above antibody, thereby forming an antibody complex. Suitable solid supports include protein binding surface, colloidal metal particles, iron oxide particles, latex particles, and polymeric beads. Moreover, by choosing appropriate antibodies, one can form a complex that precipitates from solution.

A second method measuring CIP uses a multi-antibody labeled complex. A first antibody is used similar to that in the first method, in an amount sufficient to bind the CIP present. (The first antibody can be bound to a solid support.) Again, the first antibody is allowed to bind to any CIP present, thereby forming a complex. A second antibody is added that has a label or signal generating component attached thereto and that specifically binds to a portion of CIP other than the initial peptide sequence which binds to the first antibody and allowed to bind to the CIP complex, thereby labeling the complex. The second labeled antibody can be added either sequentially or simultaneously with the first antibody. Finally, one measures the labeled complex. (One should note that the order of these two binding reactions can be reversed.)

Optionally, one can use the second labeled antibody to bind either to the mid-portion of CIP or the C-terminal portion of the CIP fragment and also add at least a third antibody which specifically binds to an epitope left open after CIP binds to the first antibody and the second antibody, thereby forming a precipitating mass. Such antibodies are known to the art. Either the second or third antibody can be bound to a solid support.

A third method for determining CIP in a sample comprises a precipitating or turbidometric immunoassay with the following three basic steps. First, one adds to the sample an antibody or antibody fragment as in the first method in an amount sufficient to bind the CIP present, however, the first antibody may be attached to a colloidal particle or moiety that can be used to detect a signal change. Again, one allows antibody to bind to any CIP present, thereby forming a complex. Finally, one measures the change in signal due to the formation of the complex.

Suitable label or signal generating components for the above assays include conventionally known chemiluminescent agents, colorimetric agents, energy transfer agents, enzymes, fluorescent agents, and radioisotopes.

The present invention also includes kits for the CIP assays. One kit contains at least two reagents, specifically a substantially pure antibody or antibody fragment specific for a peptide sequence for CIP which by virtue of the unique CIP protein conformation is available for antibody binding but this same epitope is not available for antibody binding in CAP by virtue of the unique protein conformational differences between CAP and CIP and a labeling component that binds to CIP, but not to the above antibody epitope. Optionally, this kit can contain an antibody specific for the C-terminal portion of CIP.

A second CIP assay kit comprises a first substantially pure antibody or antibody fragment specific for a peptide sequence for CIP which by virtue of the unique CIP protein conformation is available for antibody binding but this same epitope is not available for antibody binding in CAP by virtue of the unique protein conformational differences between CAP and CIP and a second antibody that binds to CIP, but not to the above first antibody domain, which is bound to a solid support. Optionally, this kit can contain an antibody specific for the C-terminal portion of CIP.

A preferred embodiment of the present invention is an immunoradiometric assay (IRMA), often referred to as a sandwich assay. Elements employed in such an assay include a capture antibody attached to a solid support and a signal antibody having a 125-I radioactive label attached thereto. Typically, one selects a capture antibody that is specific for C-terminal PTH fragments, while the label antibody is specific for the CIP domain

One would generate target antibodies (monoclonal or polyclonal) to a number of specific regions or epitopes along the length of CAP. One portion of each of these target antibodies would be labeled for detection, for example with radioactive Iodine (125-I). A CIP/CAP mixture is made comprising 1000 pg/ml of CAP and 1000 pg/ml of CIP respectively. Another portion of each of these same antibodies is attached to a solid phase, such as a small polystyrene bead, using conventional methods known to the art. Now one forms a series of permutational combinations wherein one mixes each target labeled antibody with each target capture antibody. Calibrator solution containing CAP and CIP is incubated in these combinations for a time sufficient to allow the formation of measurable complexes. One measures the labeled complexes that contain antibodies that have bound either to the CAP or CIP present in the CIP/CAP calibrator solution. Only measurable complexes are given further consideration for selection.

Two separate additional calibrator solutions are prepared. A CIP calibrator solution is prepared containing 1000 pg/ml of CIP. A CAP calibrator solution is prepared containing 1000 pg/ml of CAP. Candidate pairs of antibodies are identified from the measurable complexes formed from pairs of target labeled antibodies and target capture antibodies in the presence of the CIP/CAP calibrator solution. Each pair is incubated with the CIP calibrator solution for a time sufficient to allow the formation of measurable complexes. One measures the labeled complexes that contain antibodies that are bound to the CIP in the CIP calibrator solution. Then, each candidate pair is incubated with the CAP calibrator solution for a time sufficient to allow the formation of measurable complexes. One measures the labeled complexes that contain antibodies that have bound to the CAP present in the CAP calibrator solution. A candidate pair that detects the CIP, but not the CAP has at least one of the antibodies in the pair that binds to an epitope that differentiates between CIP and CAP due to unique protein conformational differences between CAP and CIP.

The ordinarily skilled artisan can appreciate that the present invention can incorporate any number of the preferred features described above.

All publications or unpublished patent applications mentioned herein are hereby incorporated by reference thereto.

Other embodiments of the present invention are not presented here which are obvious to those of ordinary skill in the art, now or during the term of any patent issuing from this patent specification, and thus, are within the spirit and scope of the present invention.

We claim:

1. A method for determining cyclase inhibiting parathyroid hormone (CIP) in a sample comprising:
a) adding to the sample a labeled antibody or antibody fragment specific for a peptide sequence for CIP that presents an epitope available for antibody binding in CIP, but will not bind to this same peptide sequence in cyclase activating parathyroid hormone, in an amount sufficient to bind the CIP present;
b) allowing the labeled antibody to bind to any CIP present, thereby forming a complex; and
c) measuring the amount of labeled complex.

2. The method of claim 1 wherein the labeled CIP antibody or antibody fragment is one of the following, a monoclonal antibody and a polyclonal antibody.

3. The method of claim 1 wherein a second antibody is added which is bound to a solid support and specifically binds to a portion of CIP other than that of the labeled antibody, thereby forming a complex.

4. The method of claim 3 wherein the solid support is selected from the group consisting of a protein binding surface, colloidal metal particles, iron oxide particles, latex particles, and polymeric beads.

5. The method of claim 3 wherein the complex precipitates from solution.

6. The method of claim 1 wherein the label or signal generating component is selected from the group consisting of chemiluminescent agents, colorimetric agents, energy transfer agents, enzymes, fluorescent agents, and radioisotopes.

7. A method for measuring the amount of cyclase inhibiting parathyroid hormone (CIP) fragment in a sample comprising:
a) adding to the sample a first antibody or antibody fragment specific for a peptide sequence for CIP that presents an epitope available for antibody binding in CIP, but does not bind to this same peptide sequence in cyclase activating parathyroid hormone, in an amount sufficient to bind the CIP present;
b) allowing the first antibody to bind to any CIP present, thereby forming a complex;
c) labeling the complex by means of adding a second antibody that has a label or signal generating component attached thereto and that specifically binds to a portion of CIP other than the initial peptide sequence which binds to the first antibody and allowing the second antibody to bind to the complex; and
d) measuring the amount of labeled complex.

8. The method of claim 7 wherein the second labeled antibody is added sequentially or simultaneously with the first antibody.

9. The method of claim 7 wherein the first antibody is bound to a solid support.

10. The method of claim 7 wherein the second labeled antibody binds either to the mid-portion of CIP or the C-terminal of CIP and also comprising adding at least a third antibody that specifically binds to an epitope left open after CIP binds to the first antibody and the second antibody, thereby forming a precipitating mass.

11. The method of claim 10 wherein the C-terminal CIP antibody is bound to a solid support.

12. A method for measuring cyclase inhibiting parathyroid hormone (CIP) by means of a precipitating or turbidometric immunoassay comprising:
a) adding to the sample a first antibody or antibody fragment specific for a peptide sequence for CIP that presents an epitope available for antibody binding in CIP, but does not bind to this same peptide sequence in cyclase activating parathyroid hormone, in an amount sufficient to bind the CIP present, said antibody being attached to a colloidal particle or moiety which can be used to detect a signal change;
b) allowing the antibody to bind to any CIP present, thereby forming a complex; and
c) measuring the change in signal due to the formation of the complex.

14. A substantially pure antibody or antibody fragment sample a labeled antibody or antibody fragment specific for a peptide sequence for cyclase inhibiting parathyroid hormone that comprises an epitope available for antibody binding in CIP, but does not bind to this same peptide sequence in cyclase activating parathyroid hormone.

15. The antibody of claim 14 wherein the antibody is one of the following, a monoclonal and a polyclonal antibody.

16. A kit containing reagents for performing an assay for cyclase inhibiting parathyroid hormone (CIP) comprising:
a) a substantially pure antibody or antibody fragment specific for a peptide sequence for CIP that presents an epitope available for antibody binding in CIP, but is not specific for this same peptide sequence in cyclase activating parathyroid hormone; and
b) a labeling component that binds to CIP, but not to the CIP antibody epitope bound by the first antibody.

17. The kit of claim 16 also comprising an antibody specific for the C-terminal portion of CIP.

18. A kit containing reagents for performing an assay for cyclase inhibiting parathyroid hormone (CIP) comprising:
a) a first substantially pure antibody or antibody fragment specific for a peptide sequence for CIP that presents an epitope available for antibody binding in CIP, but does not bind to this same peptide sequence in cyclase activating parathyroid hormone; and
b) a second antibody that binds to CIP, but not to the first CIP antibody epitope, which is bound to a solid support.

19. The kit of claim 18 also comprising an antibody specific for the C-terminal portion of CIP.
